(19) United States
(12) Patent Application Publication
(10) Pub. No.: US 2005/0064521 A1
(43) Pub. Date: Mar. 24, 2005

(54) In vitro assay for evaluation of angiogenic effects

(75) Inventors: Vie Cheng Yang, Taichung (TW); Yi-Jun Ye, Miaoli (TW); Jian-Jia Cheng, Hu-Kou Country (TW); Shih-Wei Kuo, Taipei (TW)

Correspondence Address: Ladas & Parry, 26 West 61st Street, New York, NY, 10023, US

(73) Assignee: TUNGHAI UNIVERSITY

(21) Appl. No.: 10/669,836

(22) Filed: Sep. 24, 2003

(51) Int. Cl. G01N 33/574; G01N 1/30

(52) U.S. Cl. 435/7.23; 435/40.5

(57) ABSTRACT

The present invention provides an in vitro method of assaying angiogenic effects, using the morphology of an in vitro model prepared by culturing endothelial cells in a three-dimensional collagen fiber gel as an index. The invention also provides a method for screening of prospective pro- and anti-angiogenic compounds.

### BACKGROUND OF THE INVENTION

1. Field of the Invention

The invention mainly relates to an in vitro method of assaying angiogenic effects. In particular, the invention provides a method for screening of prospective pro- and anti-angiogenic compounds.

2. Description of the Related Art

Angiogenesis is a morphogenic process that results in the formation of new capillary blood vessels, which is essential for embryo growth, and the progress of disease curing or wound healing. Angiogenesis is also believed to be essential for tumor or cancer growth.

Angiogenesis is an extremely complex process involving a wide range of growth factors, extracellular matrix molecules, enzymes and various cell types. Therefore, it is difficult to develop an in vitro assay that models the entire in vivo process due to the complexity of angiogenesis.

It is found that in the presence of angiogenic stimulators, vascular endothelial cells (ECs) will secret matrix metalloproteinases (MMPs) such as collagenase, gelatinase, stromulysins etc. into the extracellular matrix. The MMPs disrupt the basement membrane that encompasses ECs and allow the migration of endothelial cells into the extra-vascular space to form new capillary blood vessels. Therefore, several assay methods were developed for evaluation of angiogenic effects.

Scott et al. provided an assay for MMP-1 activity (Scott et al. A matrix metalloproteinase inhibitor which prevents fibroblast-mediated collagen lattice contraction. FEBS Letters 441, 1998, 137-140). The assay method comprised incubating the compounds to be tested with commercially available acid-soluble type I collagen from calf skin radiolabelled with [³H] acetic anhydride, determining the amount of digested collagen by measuring the radioactivity. However, the method using a radioisotope has many disadvantages, such as being expensive, difficult in operation, and harm to the environments.

Sharma et al. provided a chick chorioallantoic membrane (CAM) model and an oncogene-trasfected angiogenitic cell line for monitoring collagen lattice contraction (Sharma et al. A quantitative angiogenesis model for efficacy testing of chemopreventive agents. Anticancer Research 21, 2001, 3829-3838). The method comprised placing the tumorigenic cells held by a primary agarose pellet alone or with a secondary pellet incorporating a test agent, on an exposed CAM of 7-day-old chick embryo, and exposing the CAM by cutting a window on one side of the egg using the false airsac technique. The steps of this method are complicated and difficult in operation.

Kondo et al. revealed the combination of colloidal gold migration assay and tube formation for assaying angiogenesis (Kondo et al. Tea catechins inhibit angiogenesis in vitro, measured by human endothelial cell growth, migration and tube formation, through inhibition of VEGF receptor binding. Cancer Letters 180, 2002, 139-144). In the colloidal gold migration assay, human umbilical vein endothelial cells were plated onto over slips precoated with gelatin and colloidal gold particles; after treating the cells with tea catechins, the angiogenesis inhibitor, areas devoid of gold particles were measured by the NIH Image program. In the tube formation assay, the cells were incubated in the wells of a plate coated with the type I collagen gels, and fixed and stained the cells after treatment with Giemza™ stain solution (Merck®, Germany), and measured the areas of formed tubes by the NIH Image program. The accuracy of the assay involving an image process depends on the resolution of image uptaking.

There is a commercial kit, BD Biocoat™ Angiogenesis System for Endothelial Cell, for evaluating and quantitating endothelial cell using real-time fluorescence and non-destructive sample preparation, which was produced by BD Biosciences Discovery Labware®. The system consists of a light-tight FluoroBlok™ PET membrane that effectively blocks the transmission of light from 490 to 770 nm. The FluoroBlok™ membrane is uniformly coated with BD Matrigel™ Matrix that serves as a reconstituted authentic basement membrane in vitro. The matrix is a solubilized basement membrane preparation extracted from Engelbreth-Holm-Swarm mouse sarcoma, which contains laminin, collagen type IV, heparan sulfate proteoglycan, entactin and growth factors occurring naturally in the EHS tumor. The uniform layer of BD Matrigel™ Matrix provides an appropriate protein structure that is a true barrier to noninvasion cells, but allows invasive endothelial cells to penetrate and pass through the FluoroBlok™ membrane. Since the FluoroBlok™ membrane effectively blocks the fluorescence of labeled cells that have not invaded through the membrane, only those cells that appear on the underside of the FluoroBlok™ membrane are quantitated by fluorescence, providing a signal that can be directly correlated to cell number. However, cell invasion is not a good index for assaying angiogenesis since angiogenesis comprises not only cell invasion but also cell division and differentiation.

It is still desired to develop a quick, easy and economical method for assaying angiogenic effects.

### SUMMARY OF THE INVENTION

The invention provides a quick, easy and economical method for assaying angiogenic effects.

The invention provides an in vitro model for evaluating the effects on angiogenesis, which is prepared by culturing endothelial cells in a three-dimensional collagen fiber gel with a medium suitable for the tube formation of endothelial cells.

The invention also provides an in vitro method for assaying angiogenic effects of a compound, which comprises the steps of:

(a) providing models that prepared by culturing endothelial cells in three-dimensional collagen fiber gels with a medium suitable for the tube formation of endothelial cells;

(b) culturing the models added with and without the compound to be assayed, respectively, for a time sufficient for the tube formation of endothelial cells in the collagen fiber gels of the models; and

(c) observing the morphology of the collagen fiber gels of the models after the culturing of step (b);

(d) comparing the morphologic differences between the collagen fiber gels of the models added with the compound and those added without the compound; and determining the effect of the compound on angiogenesis by comparing the degrees of collapse of the collagen fiber gels of the models; wherein the compound is determined to have a stimulatory effect on angiogenesis in case the collapse of the collagen fiber gel of the model added with the compound is increased in comparison with that added without the compound; in contrast, the compound is determined to have an inhibitory effect on angiogenesis in case the collapse of the collagen fiber gel of the model added with the compound is decreased in comparison with that added without the compound.

### BRIEF DESCRIPTION OF THE DRAWINGS

FIG. 1 provides the inverted microscopic views of the model obtained in Example 1 after the culturing for 0 hour (a); the culturing for 6 hours (b); the culturing for 12 hours (c); the culturing for 24 hours (d); the culturing for 48 hours (e); and the culturing for 72 hours (f). (Bar=10 μm.)

FIG. 2 provides the microscopic views of the model obtained in Example 1. a: top view. b: side view. left: before the culturing. right: after the culturing.

FIG. 3 illustrates the degree of collapse of the collagen fiber gel of the model cultured with a normal medium. The values of O.D. 575 of the gel were determined by ELISA.

FIG. 4 illustrates the inhibitory effects of the collapse of the collagen fiber gels of the models by tea catechin (EGCg); wherein the models were cultured with 25 μM EGCg (a); 25 μM EGCg (b); and 25 μM EGCg (c), respectively.

FIG. 5 illustrates the inhibitory effects of the collapse between the models obtained in Example 2 by Thalidomide. The gels were subject to ELISA to determine the values of O.D. 575.

### DETAILED DESCRIPTION OF THE INVENTION

As used herein, the term “angiogenesis” refers to a process in which a network of new blood vessels emerges from preexisting vessels. The “effect on angiogenesis” refers to the effect in stimulating or inhibiting angiogenesis.

The invention provides an in vitro model for evaluating the effects on angiogenesis, which is prepared by culturing endothelial cells in a three-dimensional collagen fiber gel with a medium suitable for the tube formation of endothelia cells.

According to the invention, the three-dimensional collagen fiber gel plays the role of the extracellular matrix. During the angiogenesis, the MMPs digest and collapse the extracellular matrix to gain space for new blood vessels growing. Therefore, the degrees of collapse of the extracellular matrix (i.e., the collagen fiber gel) can be used as an index for evaluating the effects on angiogenesis. The three-dimensional collagen fiber gel according to the invention provides a framework for the growth of endothelial cells and formation of tubular structures. Any natural or artificial collagen fiber gel showing a significant difference between before and after angiogenesis may be used in the invention. In one preferred embodiment of the invention, the collagen is type I collagen.

In one embodiment of the invention, the three-dimensional collagen fiber gel is prepared by dissolving a dry collagen fiber in a solution, and exposing it at a low temperature for solidification. The dry collagen fiber may be obtained from animal or by genetic engineering technology. The dry collagen fiber is dissolved in a solution and then exposed at a low temperature (preferably at a temperature ranging from about 4 to about 10° C.) for solidification to provide a framework for the growth of endothelial cells. In one preferred embodiment of the invention, the dry collagen fiber gel is obtained from the tails of rats and the solution is an acetic acid solution.

According to the invention, the endothelial cells may be blood vessel endothelial cells including artery and vein endothelial cells. The choice of artery or vein endothelial cells depends on what kind of blood vessel angiogenesis to be observed. Preferably, the endothelial cells are derived from mammals. More preferably, the endothelial cells are derived from human.

According to the invention, the endothelial cells are cultured in the three-dimensional collagen fiber gel in a medium suitable for the formation of tubular structures of endothelial cells in the three-dimensional collagen fiber gel. The medium may be added with growth factors and alkaloid for tube formation. In a preferred embodiment in the invention, the growth factors may be angiogenic stimulators, including vascular endothelial growth factor (VEGF), epigallocatechin gallate (EGCg), and tumor necrosis factor-α (TNF-α); and the alkaloid is preferably 12-o-teradecanoyl phorbol 13-acetate (TPA). The medium may be conditioned for evaluation of angiogenesis under a specific condition. For example, a tumor cell culture broth may be used as the medium for investigating the angiogenesis in a tumor.

In the model of the invention, the endothelial cells have the intensity to secrete MMPs to digest the collagen and differentiate into tubular structures in the three-dimensional collagen fiber gel.

The invention also provides an in vitro method for assaying angiogenic effects of a compound, which comprises the steps of:

(a) providing models that prepared by culturing endothelial cells in three-dimensional collagen fiber gels with a medium suitable for the tube formation of endothelial cells;

(b) culturing the models added with and without the compound to be assayed, respectively, for a time sufficient for the tube formation of the endothelial cells in the collagen fiber gels of the models; and

(c) observing the morphology of the collagen fiber gels of the models after the culturing of step (b);

(d) comparing the morphologic differences between the collagen fiber gels of the models added with the compound and those added without the compound; and determining the effect of the compound on angiogenesis by comparing the degrees of collapse of the collagen fiber gels of the models; wherein the compound is determined to have a stimulatory effect on angiogenesis in case the collapse of the collagen fiber gel of the model added with the compound is increased in comparison with that added without the compound; in contrast, the compound is determined to have an inhibitory effect on angiogenesis in case the collapse of the collagen fiber gel of the model added with the compound is decreased in comparison with that added without the compound.

According to the invention, the endothelial cells are cultured in the collagen fiber gels of the models of the invention. The cultures are added with and without the compound to be assayed, respectively, for a time sufficient for the tube formation of the endothelial cells in the collagen fiber gels of the models. According to the invention, the models are cultured with and without the compound to be assayed, respectively, for a time sufficient for the tube formation of the endothelial cells in the collagen fiber gels of the models, such as 6 to 24 hours. In one preferred embodiment of the invention, it takes only 12 hours to conduct the method. Then, the morphology of the three-dimensional collagen fiber gel is observed for determining the degrees of collapse, including the thickness or amount of the three-dimensional collagen fiber gel and the pattern of tubular structure formed in the gel.

The morphologic differences between the three-dimensional collagen fiber gels of the models added with the compound and those added without the compound may be compared by the method comprising the following steps of:

(1) removing the medium of the models;

(2) staining the three-dimensional collagen fiber gels of the models with a dye; and

(3) quantifying and comparing the absorbance of the dye in the models.

As used herein, the term “dye” refers to a compound with the ability to stain the three-dimensional collagen fiber gel and whose absorbance can be detected and quantified at a specific wave-length. In one preferred embodiment of the invention, the dye is trypan blue.

The effect of a compound on angiogenesis is determined by comparing the degrees of collapse of the collagen fiber gels of the models. The compound is determined to have a stimulatory effect on angiogenesis in case the collapse of the collagen fiber gel of the model added with the compound is increased in comparison with that added without the compound; in contrast, the compound is determined to have an inhibitory effect on angiogenesis in case the collapse of the collagen fiber gel of the model added with the compound is decreased in comparison with that added without the compound. The collapse of the collagen fiber gels may be determined by any conventional method known to persons skilled in the art; e.g., the measure of the thickness or absorbance of the collagen fiber gels. For instance, if the thickness of the three-dimensional collagen fiber gel in the models added with the compound is lower than that added without the compound, or if the absorbance of the three-dimensional collagen fiber gel of the model added with the compound after staining with a dye is lower than that added without the compound, the compound is determined to have a stimulatory effect on angiogenesis. In contrast, if the thickness of the three-dimensional collagen fiber gel in the models added with the compound is higher than that added without the compound, or if the absorbance of the three-dimensional collagen fiber gel of the model added with the compound after staining with a dye is higher than that added without the compound, the compound is determined to have an inhibitory effect on angiogenesis.

The present invention provides a good model and an in vitro method for assaying and quantitating the effects on angiogenesis. It takes only about 12 to about 24 hours to conduct the method of the invention, which is much quicker than the conventional in vitro methods, which it normally takes about one week to conduct. No complicated steps and no hazard substances are required to conduct the method of the invention. Besides, the costs for performing the method are also low, and the quantitation or evaluation of the effects on angiogenesis by measuring thickness or absorbance is very easy.

The method according to the invention may be widely applied for assessment of the angiogenic potential; in particular, the method may be used for screening prospective pro- and anti-angiogenic compounds, or screening new drugs for modulating angiogenesis.

The following Examples are given for the purpose of illustration only and are not intended to limit the scope of the present invention.

### EXAMPLE 1

### In vitro Model

Three-dimensional collagen fiber gel: The dry collagen fiber was extract from tails of Sprague-Dawley (SD) rats. The fiber was dissolved in 1 N acetic acid solution in the concentration of 1 g fiber/100 mL acetic acid. The dissolving was performed at 4° C. for 48 hours and followed by dialysis three times with 0.25 N acetic acid solution for 24 hours.

Endothelial cells: SD rats weighting 100 to 200 g were anaesthetized with 0.1 mL/100 g body weight of barbiturate (Nembutal™, Abbott® Laboratories, USA) intraperitoneally. The thoracic aortas were cut and the connective tissues were removed. The vessels were further split into vessel rings thickening about 2 mm, and then cultured the rings in fresh Dulbecco's modified Eagle medium (DMEM) with 10% fetal bovine serum. The culture was maintained at 37° C. with 5% CO₂ with changing the medium every two days. After cultured for 5 to 6 days, the cells were harvested. The endothelial cells suitable for the method according to the invention were the second to tenth passage cells.

Model: The endothelial cells (3×10⁶) were mixed in the three-dimensional collagen fiber gel and cultured in DMEM containing 5% fetal bovine serum and 10 ng/mL VEGF and 3.08 ng/mL TPA at 37° C. After culturing the endothelial cells for 0, 6, 12, 24, 48, and 72 hours, the morphologies of the cell and gel were both observed with an inverted microscope.

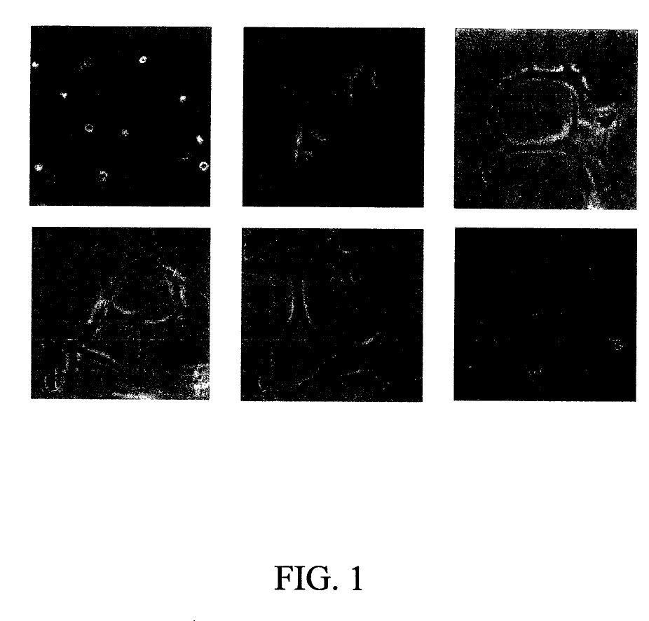

The inverted microscopic views of the angiogenesis model culturing for 0, 6, 12, 24, 48, and 72 hours were shown in FIG. 1. When culturing for 0 hour (FIG. 1a), the cells were not attached and showed as granular light spots. When culturing for 6 hours (FIG. 1b), the cells started to change and small amount of tube appeared. When culturing for 12 hour (FIG. 1a), significant tubes were formed. With the duration of culture increasing, the tubes were more significant (FIG. 1d, e, and f).

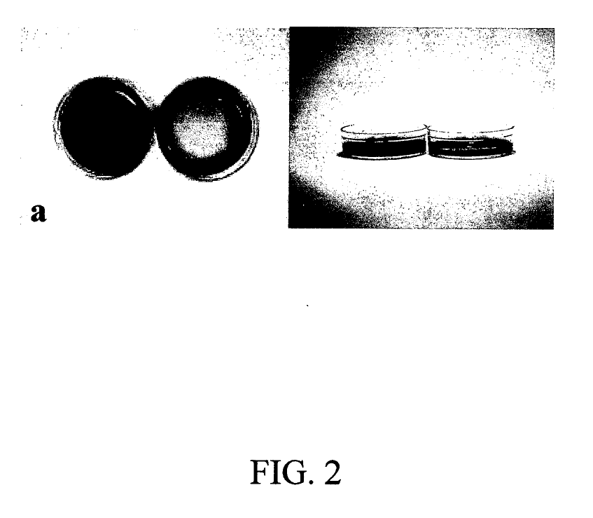

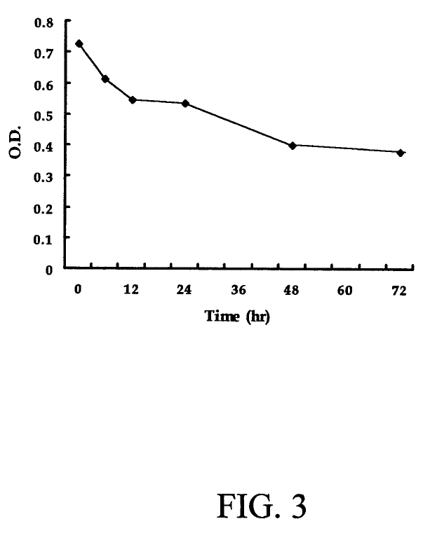

After culturing for 48 hours, the medium was removed and 0.075% trypan blue was added for staining the gels for 30 minutes. The upper solution was then removed and the absorbance of the solution was determined with a spectroscope. After culturing, the endothelial cells secreted MMPs to digest the three-dimensional collagen fiber gel in the proliferation and differentiation. Therefore, the three-dimensional collagen fiber gel became thinner (referring to FIGS. 2a and 2b, right) than that before culturing (referring to FIGS. 2a and 2b, left). The O.D. values measured at 450 nm showed the same result (referring to FIG. 3).

### Example 2

### Assay for Angiogenesis Inhibitor Using the Angiogenesis Model

Assay: The models as obtained in Example 1 were added with 25 μM, 50 μM and 100 μM of ECGg (an angiogenesis inhibitor), respectively. The results were obtained by repeating several times of the assay.

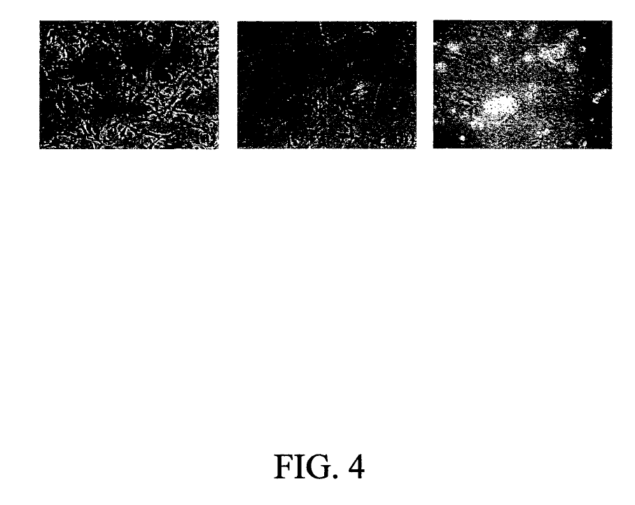

Results: The inverted microscopic views of the models cultured for 48 hours were shown in FIG. 4. The model added with 25 μM EGCg showed to have less tubes and lower cell count (referring to FIG. 4a) than those added without EGCg (as a control), see FIG. 4c. The model added with 100 μM EGCg showed to have almost no tubes, see FIG. 4b.

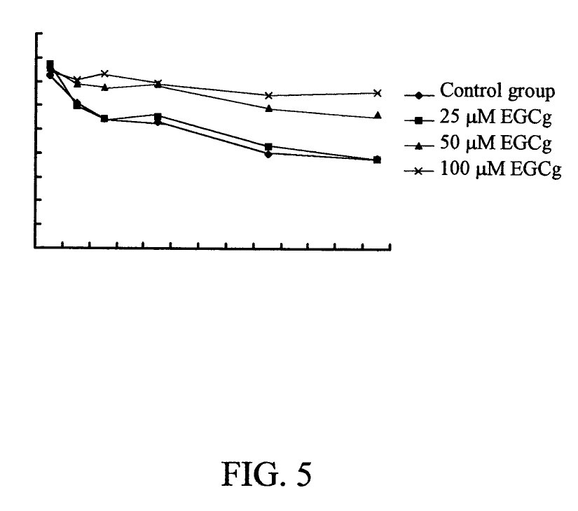

The results of the absorbance measurement were given in FIG. 5. It showed that the model added with 25 μM EGCg had a little inhibitory effect on angiogenesis. In the model added with 50 μM EGCg, the collapse of the gel was nearly totally inhibited. In the model added with 100 μM EGCg, nearly no collapse was observed. It was evidenced that the degree of collapse of the three-dimensional collagen fiber gel is decreased with the addition of EGCg. That results support that the effect of a compound on angiogenesis can be determined by comparing the degrees of collapse of the collagen fiber gels of the models. For instance, the compound is determined to have a stimulatory effect on angiogenesis in case the collapse of the collagen fiber gel of the model added with the compound is increased in comparison with that added without the compound; in contrast, the compound is determined to have an inhibitory effect on angiogenesis in case the collapse of the collagen fiber gel of the model added with the compound is decreased in comparison with that added without the compound.

While embodiments of the present invention have been illustrated and described, various modifications and improvements can be made by persons skilled in the art. It is intended that the present invention is not limited to the particular forms as illustrated, and that all the modifications not departing from the spirit and scope of the present invention are within the scope as defined in the appended claims.

1. An in vitro model for evaluating the effects on angiogenesis, which is prepared by culturing endothelial cells in a three-dimensional collagen fiber gel with a medium suitable for the tube formation of endothelial cells.

2. The model according to claim 1, wherein the collagen is type I collagen.

3. The model according to claim 1, wherein the three-dimensional collagen fiber gel is obtained by dissolving a dry collagen fiber in a solution at the temperature of 4 to 10° C.

4. The model according to claim 3, wherein the solution is an acetic acid solution.

5. The model according to claim 1, wherein the endothelial cells is derived from mammals.

6. The model according to claim 5, wherein the mammals is human.

7. The model according to claim 1, wherein the medium comprises growth factors, such as vascular endothelial growth factor (VEGF), epigallocatechin gallate (EGCg), and tumor necrosis factor-α (TNF-α).

8. The model according to claim 1, wherein the medium comprises alkaloid, such as 12-o-teradecanoyl phorbol 13-acetate (TPA).

9. The model according to claim 1, wherein the medium comprises optionally a tumor cell culture broth.

10. An in vitro method for assaying angiogenic effects of a compound, which comprises the steps of:
(a) providing the model according to claim 1;
(b) culturing the models added with and without the compound to be assayed, respectively, for a time sufficient for the tube formation of endothelial cells in the collagen fiber gels of the models; and
(c) observing the morphology of the collagen fiber gels of the models after the culturing of step (b);
(d) comparing the morphologic differences between the collagen fiber gels of the models added with the compound and those added without the compound; and determining the effect of the compound on angiogenesis by comparing the degrees of collapse of the collagen fiber gels of the models; wherein the compound is determined to have a stimulatory effect on angiogenesis in case the collapse of the collagen fiber gel of the model added with the compound is increased in comparison with that added without the compound; in contrast, the compound is determined to have an inhibitory effect on angiogenesis in case the collapse of the collagen fiber gel of the model added with the compound is decreased in comparison with that added without the compound.

11. The method according to claim 10, wherein the collagen is type I collagen.

12. The method according to claim 10, wherein the three-dimensional collagen fiber gel in the step (a) is obtained by dissolving a dry collagen fiber in a solution at the temperature of 4 to 10° C.

13. The method according to claim 12, wherein the solution is an acetic acid solution.

14. The method according to claim 10, wherein the endothelial cells in the step (a) is derived from mammals.

15. The method according to claim 14, wherein the mammals is human.

16. The method according to claim 10, wherein the medium comprises growth factors, such as vascular endothelial growth factor (VEGF), epigallocatechin gallate (EGCg), and tumor necrosis factor-α (TNF-α).

17. The method according to claim 10, wherein the medium comprises alkaloid, such as 12-o-teradecanoyl phorbol 13-acetate (TPA).

18. The model according to claim 10, wherein the medium comprises optionally a tumor cell culture broth.

19. The model according to claim 10, wherein the morphology of the three-dimensional collagen fiber gels in the step (c) is compared by measuring the thickness of the three-dimensional collagen fiber gels.

20. The method according to claim 10, wherein the morphology of the three-dimensional collagen fiber gels in the step (c) is compared by an absorbance measurement.

21. The method according to claim 20, wherein the absorbance measurement comprises the steps of:
(1) removing the medium of the models;
(2) staining the three-dimensional collagen fiber gels of the models with a dye; and
(3) quantifying and comparing the absorbance of the dye in the models.

22. The method according to claim 21, wherein the dye is trypan blue.
